(19) United States
(12) Patent Application Publication
(10) Pub. No.: US 2024/0093146 A1
(43) Pub. Date: Mar. 21, 2024

(54) METHOD FOR PRODUCING OLIGODENDROCYTES

(71) Applicants: Koji TANABE, Palo Alto, CA (US); I Peace, Inc., Palo Alto, CA (US)

(72) Inventor: Koji TANABE, Palo Alto, CA (US)

(73) Assignees: Koji TANABE, Palo Alto, CA (US); I Peace, Inc., Palo Alto, CA (US)

(21) Appl. No.: 18/258,778

(22) PCT Filed: Jan. 19, 2022
(86) PCT No.: PCT/JP2022/001814

§ 371 Date: Jun. 21, 2023

Related U.S. Application Data
(60) Provisional application No. 63/141,626, filed on Jan. 26, 2021.

(51) Int. Cl. C12N 5/079

(52) U.S. Cl. CPC C12N 5/0622; C12N 2506/11; C12N 2506/45

(57) ABSTRACT

According to the present disclosure, there is provided a method for producing oligodendrocytes and progenitor cells thereof, comprising introducing inducers including a combination of OLIG and SOX or a combination of OLIG and NKX into iPS or ES cells. According to the present disclosure, there is also provided a method for producing oligodendrocytes and progenitor cells thereof, comprising introducing inducers including OLIG, SOX, ASCL, and NKX into somatic cells.

### TECHNICAL FIELD

The present invention relates to cell technology and methods for producing oligodendrocytes and progenitor cells thereof.

### BACKGROUND ART

Oligodendrocytes, which are myelinating cells of the central nervous system, may be useful for cell transplantation for the treatment of hereditary and acquired leukodystrophy (see, e.g., Patent Documents 1 and 2). However, conventional methods of inducing oligodendrocytes from stem cells have problems of time consuming and inefficiency.

### CITATION LIST

### Patent Documents

Patent Document 1: Japanese Patent No. 6541577
Patent Document 2: International Publication No. 2011/091048

### SUMMARY OF INVENTION

### Technical Problem

The present invention, in part, aims to provide methods for efficiently producing oligodendrocytes and progenitor cells thereof.

### Solution to Problem

According to an aspect of the present invention, there is provided a method for producing oligodendrocytes and progenitor cells thereof, comprising introducing inducers including OLIG and SOX into iPS or ES cells.

According to an aspect of the present invention, there is provided a method for producing oligodendrocytes and progenitor cells thereof, comprising introducing inducers including OLIG and NKX into iPS or ES cells.

According to an aspect of the present invention, there is provided a method for producing oligodendrocytes and progenitor cells thereof, comprising introducing inducers including OLIG, NKX, and SOX into iPS or ES cells.

In the methods for producing oligodendrocytes and progenitor cells thereof, the inducers may further include a factor that promotes cell proliferation.

In the methods for producing oligodendrocytes and progenitor cells thereof, the factor that promotes cell proliferation may be at least one selected from the group consisting of a factor that suppresses p53 gene, a factor that suppresses Rb gene, and MYC. MYC may be c-MYC.

In the methods for producing oligodendrocytes and progenitor cells thereof, the inducers may further include ASCL.

According to an aspect of the present invention, there is also provided a method for producing oligodendrocytes and progenitor cells thereof, comprising introducing inducers including OLIG and a factor that promotes cell proliferation into iPS or ES cells.

In the method for producing oligodendrocytes and progenitor cells thereof, the factor that promotes cell proliferation may be at least one selected from the group consisting of a factor that suppresses p53 gene, a factor that suppresses Rb gene, and MYC. MYC may be c-MYC.

In the method for producing oligodendrocytes and progenitor cells thereof, the inducers may include at least any selected from the group consisting of SOX, ASCL, and NKX.

According to an aspect of the present invention, there is also provided a method for producing oligodendrocytes and progenitor cells thereof, comprising producing iPS cells without cloning cells that have been introduced with reprogramming factors and introducing an inducer including OLIG into the iPS cells.

According to an aspect of the present invention, there is also provided a method for producing oligodendrocytes and progenitor cells thereof, comprising producing iPS cells by seeding cells that have been introduced with reprogramming factors, without cloning them and introducing an inducer including OLIG into the iPS cells.

According to an aspect of the present invention, there is also provided a method for producing oligodendrocytes and progenitor cells thereof, comprising producing iPS cells by detaching cells that have been introduced with reprogramming factors from a culture vessel, mixing together at least some of the detached cells, and seeding them, and introducing an inducer including OLIG into the iPS cells.

According to an aspect of the present invention, there is also provided a method for producing oligodendrocytes and progenitor cells thereof, comprising producing iPS cells by collecting cells that have been introduced with reprogramming factors from a culture vessel, mixing together at least some of the collected cells, and seeding them, and introducing an inducer including OLIG into the iPS cells.

According to an aspect of the present invention, there is also provided a method for producing oligodendrocytes and progenitor cells thereof, comprising producing iPS cells without picking up each of a plurality of colonies formed by cells that have been introduced with reprogramming factors and introducing an inducer including OLIG into the iPS cells.

According to an aspect of the present invention, there is also provided a method for producing oligodendrocytes and progenitor cells thereof, comprising producing iPS cells by mixing together cells that have been introduced with reprogramming factors and are derived from different single cells and seeding them, and introducing an inducer including OLIG into the iPS cells.

In the seeding in the methods for producing oligodendrocytes and progenitor cells thereof, the cells are not required to be cloned.

In the seeding in the methods for producing oligodendrocytes and progenitor cells thereof, the cells that have been introduced with reprogramming factors may be mixed with each other.

In the seeding in the methods for producing oligodendrocytes and progenitor cells thereof, clones derived from cells that have been introduced with reprogramming factors may be mixed with each other.

In the seeding in the methods for producing oligodendrocytes and progenitor cells thereof, different clones derived from cells that have been introduced with reprogramming factors may be mixed with each other.

The methods for producing oligodendrocytes and progenitor cells thereof are not required to comprise separating a plurality of colonies formed by the cells that have been introduced with reprogramming factors from each other before seeding them.

In the methods for producing oligodendrocytes and progenitor cells thereof, each of the plurality of colonies formed by the cells that have been introduced with reprogramming factors is not required to be picked up before seeding them.

In the seeding in the methods for producing oligodendrocytes and progenitor cells thereof, the plurality of colonies formed by the cells that have been introduced with reprogramming factors may be mixed with each other.

The methods for producing oligodendrocytes and progenitor cells thereof are not required to comprise cloning a single colony formed by the cells that have been introduced with reprogramming factors.

The methods for producing oligodendrocytes and progenitor cells thereof are not required to comprise picking up a colony formed by the cells that have been introduced with reprogramming factors.

In the methods for producing oligodendrocytes and progenitor cells thereof, the cells that have been introduced with reprogramming factors and have attached onto a culture vessel may be collected to seed at least some of the collected cells into a medium.

In the methods for producing oligodendrocytes and progenitor cells thereof, the cells that have been introduced with reprogramming factors may be seeded without distinguishing the cells based on their gene expression states.

In the methods for producing oligodendrocytes and progenitor cells thereof, the cells that have been introduced with reprogramming factors may be seeded without distinguishing the cells based on the degree of reprogramming.

In the methods for producing oligodendrocytes and progenitor cells thereof, the inducer may include at least any selected from the group consisting of SOX, ASCL, and NKX.

According to an aspect of the present invention, there is also provided a method for producing oligodendrocytes and progenitor cells thereof, comprising introducing inducers including OLIG, SOX, ASCL, and NKX into somatic cells.

In the methods for producing oligodendrocytes and progenitor cells thereof, the inducers may further include a factor that promotes cell proliferation.

According to an aspect of the present invention, there is also provided a method for producing oligodendrocytes and progenitor cells thereof, comprising introducing inducers including OLIG and a factor that promotes cell proliferation, into somatic cells.

In the method for producing oligodendrocytes and progenitor cells thereof, the inducers may include at least any selected from the group consisting of SOX, ASCL, and NKX.

In the method for producing oligodendrocytes and progenitor cells thereof, the factor that promotes cell proliferation may be at least one selected from the group consisting of a factor that suppresses p53 gene, a factor that suppresses Rb gene, and MYC. MYC may be c-MYC.

In the method for producing oligodendrocytes and progenitor cells thereof, the somatic cells are not required to be iPS or ES cells.

In the method for producing oligodendrocytes and progenitor cells thereof, the somatic cells may be blood cells.

In the method for producing oligodendrocytes and progenitor cells thereof, the somatic cells may be mononuclear cells.

In the method for producing oligodendrocytes and progenitor cells thereof, the somatic cells may be fibroblast cells.

### Advantageous Effects of Invention

The present invention can provide efficient methods for producing oligodendrocytes and progenitor cells thereof.

### BRIEF DESCRIPTION OF DRAWINGS

FIG. 1 shows graphs of the results by a flow cytometer according to Example 1.

FIG. 2 shows graphs of the results of PCR according to Example 1.

FIG. 3 shows images of TRA1-60-positive cells according to Example 1.

FIG. 4 shows graphs of the clonal efficiency according to Examples 1 and 2.

FIG. 5 shows a fluorescent microscope image of a cell according to Example 3.

FIG. 6 shows fluorescent microscope images of cells according to Example 3.

FIG. 7 shows fluorescent microscope images of cells according to Example 4.

FIG. 8 shows fluorescent microscope images of cells according to Examples 5 to 9.

FIG. 9 shows fluorescent microscope images of cells according to Example 10.

FIG. 10 shows fluorescent microscope images of cells according to Example 11.

FIG. 11 shows a fluorescent microscope image of cell according to Example 12.

FIG. 12 shows a graph of the results of Examples 13 to 18.

### DESCRIPTION OF EMBODIMENTS

Embodiments of the present invention will be described in detail below. The embodiments described below exemplify materials, substances, chemicals, methods, and the like to embody the technical concept of the present invention. The technical concept of the present invention is not limited to combinations of components and the like as described below. The technical concept of the present invention may be modified in various manners within the scope of the claims.

A method for producing oligodendrocytes and progenitor cells thereof according to an embodiment comprises introducing inducers including OLIG, SOX, ASCL, and NKX into somatic cells.

The method for producing oligodendrocytes and progenitor cells thereof according to an embodiment also comprises introducing inducers including OLIG and SOX into induced pluripotent stem (iPS) cells or embryonic stem (ES) cells.

The method for producing oligodendrocytes and progenitor cells thereof according to an embodiment also comprises introducing inducers including OLIG and NKX into iPS or ES cells.

The method for producing oligodendrocytes and progenitor cells thereof according to an embodiment also comprises producing iPS cells without cloning cells that have been introduced with reprogramming factors and introducing an inducer including OLIG into the iPS cells.

The method for producing oligodendrocytes and progenitor cells thereof according to an embodiment also comprises producing iPS cells by seeding cells that have been introduced with reprogramming factors without cloning them and introducing an inducer including OLIG into the iPS cells.

The method for producing oligodendrocytes and progenitor cells thereof according to an embodiment also comprises producing iPS cells by detaching cells that have been introduced with reprogramming factors from a culture vessel, mixing together at least some of the detached cells, and seeding them, and introducing an inducer including OLIG into the iPS cells.

The method for producing oligodendrocytes and progenitor cells thereof according to an embodiment also comprises producing iPS cells by collecting cells that have been introduced with reprogramming factors from a culture vessel, mixing together at least some of the collected cells, and seeding them, and introducing an inducer including OLIG into the iPS cells.

The method for producing oligodendrocytes and progenitor cells thereof according to an embodiment also comprises producing iPS cells without picking up each of a plurality of colonies formed by cells that have been introduced with reprogramming factors and introducing an inducer including OLIG into the iPS cells.

The method for producing oligodendrocytes and progenitor cells thereof according to an embodiment also comprises producing iPS cells by mixing together cells that have been introduced with reprogramming factors and are derived from different single cells and seeding them, and introducing an inducer including OLIG into the iPS cells.

The inducers may include OLIG and ASCL. The inducers may include OLIG, ASCL, and SOX. The inducer may include OLIG, SOX, ASCL, and NKX.

OLIG is OLIG2, for example. SOX is SOX10, for example. ASCL is ASCL1, for example. NKX is NKX2.2 or NKX6.1, for example.

The inducers may further include a factor that promotes cell proliferation. Examples of the factor that promotes cell proliferation include a factor that suppresses p53 gene, a factor that suppresses Rb gene, and MYC. The factor that promotes cell proliferation may be a factor that promotes carcinogenesis, a factor that suppresses apoptosis, or a factor that induces change of chromatin to euchromatin.

p53 is a tumor suppressor protein. The factor that suppresses p53 gene is, for example, a p53 dominant-negative mutant. The p53 dominant-negative mutant is not particularly limited as long as it can competitively acts with wild-type p53 protein present in somatic cells to inhibit the function of wild-type p53 protein. Examples of the p53 dominant-negative mutant include p53P275S, which has a point mutation of proline at position 275 (position 278 in human) located in the DNA-binding region of mouse p53 to serine; p53DD, which has deleted amino acids at positions 14 to 301 of mouse p53 (corresponding to positions 11 to 304 of human p53); p53S58A, which has a point mutation of serine at position 58 of mouse p53 (at position 61 in human) to alanine; p53C135Y, which has a point mutation of cysteine at position 135 of human p53 (at position 132 in mouse) to tyrosine; p53A135V, which has a point mutation of alanine at position 135 of mouse p53 (at position 138 in human) to valine; p53R172H, which has a point mutation of arginine at position 172 of mouse p53 (at position 175 in human) to histidine; p53R270H, which has a point mutation of arginine at position 270 of mouse p53 (at position 273 in human) to histidine; and p53D278N, which has a point mutation of aspartic acid at position 278 of mouse p53 (at position 281 in human) to asparagine. Alternatively, the factor that suppresses p53 gene may be RNA that interferes with p53 gene such as short hairpin RNA (shRNA) and siRNA.

Rb is a tumor suppressor protein. The factor that suppresses Rb gene may be, for example, RNA that interferes with Rb gene such as short hairpin RNA (shRNA) and siRNA.

MYC causes loss of the function of controlling cell cycle to induce carcinogenesis. MYC may be c-MYC.

The inducers may be DNA or RNA. The RNA may be mRNA. Although gene symbols denoted herein are represented with human gene symbols, it is not intended to limit the species by uppercase or lowercase letters. For example, even if a gene symbol is denoted with uppercase letters only, it does not exclude the inclusion of mouse or rat genes. In the Examples, however, the gene symbols corresponding to the species actually used are represented.

The somatic cells are, for example, cells that are not stem cells. The somatic cells are, for example, cells that are neither iPS cells nor ES cells. Examples of the somatic cells include fibroblast cells, blood cells, dental pulp stem cells, keratinocytes, dermal papilla cells, dental epithelial cells, and somatic stem cell progenitors. Examples of the blood cells include T cells and blood cells except T cells (non-T cells) such as macrophages, monocytes, mononuclear cells, B cells, and non-rosette-forming cells. The somatic cells may be cells found in urine. Examples of the cells found in urine include bladder epithelial cells.

The somatic cells may be derived from human or non-human animals. The somatic cells may be derived from a single individual of human or multiple individuals of human. The somatic cells may be derived from a single individual of non-human animals or multiple individuals of non-human animals. The somatic cells may be derived from a fetus.

The iPS and ES cells may be derived from human or non-human animals. The iPS and ES cells may be derived from a single individual of human or multiple individuals of human. The iPS and ES cells may be derived from a single individual of non-human animals or multiple individuals of non-human animals. The iPS cells may be derived from a fetus.

The iPS cells may be induced by, for example, a culturing method comprising culturing cells that have been introduced with reprogramming factors, collecting the cells that have been introduced with reprogramming factors, and seeding at least some of the collected cells into a medium to passage them.

The cells to be introduced with reprogramming factors are not particularly limited. Examples of the cells include fibroblast cells, blood cells, dental pulp stem cells, keratinocytes, dermal papilla cells, dental epithelial cells, and somatic stem cell progenitors. The cells to be introduced with reprogramming factors may be cells found in urine. Examples of the cells found in urine include bladder epithelial cells. The cells to be introduced with reprogramming factors may be derived from human or non-human animals. The cells to be introduced with reprogramming factors may be derived from a single individual of human or multiple individuals of human. The cells to be introduced with reprogramming factors may be derived from a single individual of non-human animals or multiple individuals of non-human animals.

Examples of the reprogramming factors to be introduced into the cells include RNA. The RNA is mRNA, for example. Examples of the reprogramming factors to be introduced into cells include RNA of OCT such as OCT3/4, RNA of SOX such as SOX2, RNA of KLF such as KLF4, and RNA of MYC such as c-MYC. The RNAs of the reprogramming factors may be M30, which is modified OCT3/4. The RNAs of the reprogramming factors may further include RNA of at least one factor selected from the group consisting of LIN28A, FOXH1, LIN28B, GLIS1, p53-dominant negative, p53-P275S, L-MYC, NANOG, DPPA2, DPPA4, DPPA5, ZIC3, BCL-2, E-RAS, TPT1, SALL2, NAC1, DAX1, TERT, ZNF206, FOXD3, REX1, UTF1, KLF2, KLF5, ESRRB, miR-291-3p, miR-294, miR-295, NR5A1, NR5A2, TBX3, MBD3sh, TH2A, TH2B, and P53DD. These RNAs are available from TriLink.

p53 is a tumor suppressor protein. The p53 dominant-negative mutant is not particularly limited as long as it can competitively acts with wild-type p53 protein present in somatic cells to inhibit the function of wild-type p53 protein. Examples of the p53 dominant-negative mutant include p53P275S, which has a point mutation of proline at position 275 (position 278 in human) located in the DNA-binding region of mouse p53 to serine; p53DD, which has deleted amino acids at positions 14 to 301 of mouse p53 (corresponding to positions 11 to 304 of human p53); p53S58A, which has a point mutation of serine at position 58 of mouse p53 (at position 61 in human) to alanine; p53C135Y, which has a point mutation of cysteine at position 135 of human p53 (at position 132 in mouse) to tyrosine; p53A135V, which has a point mutation of alanine at position 135 of mouse p53 (at position 138 in human) to valine; p53R172H, which has a point mutation of arginine at position 172 of mouse p53 (at position 175 in human) to histidine; p53R270H, which has a point mutation of arginine at position 270 of mouse p53 (at position 273 in human) to histidine; and p53D278N, which has a point mutation of aspartic acid at position 278 of mouse p53 (at position 281 in human) to asparagine.

The RNA may be modified with pseudouridine (Ψ) or 5-methyluridine (5meU). The RNA may be polyadenylated.

The RNA to be introduced into the cells is, for example, single-stranded RNA and may be substantially free of double-stranded RNA. The RNA to be introduced into the cells is preferably substantially free of short RNA and impurities such as foreign substances. To substantially remove double-stranded RNA, single-stranded RNA to be introduced into the cells may be purified and/or concentrated. Methods of purifying single-stranded RNA to be introduced into the cells include a purification method by high-performance liquid chromatography (HPLC). For example, HPLC removes 70% or more, 75% or more, 80% or more, 85% or more, or 90% or more of double-stranded RNA. Alternatively, to substantially remove double-stranded RNA, the RNA to be introduced into the cells may be treated with a ribonuclease that degrades double-stranded RNA.

The RNA to be introduced into cells may further include RNA of the transcriptional activation domain (TAD) of MYOD directly connected to the full length RNA of OCT3/4.

The reprogramming factors are introduced into the cells by, for example, lipofection. The lipofection refers to a method comprising forming a complex of nucleic acid, which is a negatively charged substance, and positively charged lipid through electrical interaction and causing incorporation of the complex into cells through endocytosis or membrane fusion. The lipofection has the advantage of less damage to cells, high introduction efficiency, simple operation, and less time-consuming.

The reprogramming factors are introduced into cells in culture using, for example, an RNA transfection reagent. For example, when the cells are mononuclear cells, RNA may be introduced into the mononuclear cells immediately after being isolated from blood.

Alternatively, the reprogramming factors are introduced into cells using, for example, a viral vector. The viral vector may be an RNA viral vector. The RNA viral vector may be a Sendai virus vector. The Sendai virus vector may be a temperature-sensitive Sendai virus vector in which the stability of the viral nucleic acid decreases at a predetermined temperature or higher. The viral nucleic acid of the temperature-sensitive Sendai virus vector is stable at a temperature lower than the predetermined temperature. The viral nucleic acid may be viral DNA or viral RNA. The viral nucleic acid may be a viral genome. The decrease in the stability of the viral nucleic acid may be at least one of degradation of the viral nucleic acid and suppression of replication or proliferation of the viral nucleic acid. Decreasing the stability of the viral nucleic acid leads to a decrease in at least one of proliferation of the viral nucleic acid and the replication rate and gene expression level of the viral nucleic acid. The predetermined temperature is, for example, a temperature equal to or higher than 36.5° C. and equal to or lower than 37.5° C. The stability, that is, at least one of proliferation, replication rate, and gene expression level, of the viral nucleic acid of temperature-sensitive Sendai virus vectors is high below a predetermined temperature and low above a predetermined temperature. For example, a proliferation rate or gene expression level of the temperature-sensitive Sendai virus vector in cells cultured at 37° C. is ½ or less of that in cells cultured at 32° C.

Sendai virus encodes N gene, P gene, M gene, F/HN gene, and L gene. HN protein recognizes sialic acid on the cell surface and tethers the viral particles to a cell when Sendai virus attaches to the cell. F protein is activated upon its cleavage by extracellular proteases to catalyze the fusion of the tethered envelope of Sendai virus with the cell membrane of the target cell to establish infection. L protein, together with its modifier protein, P protein, catalyzes replication of the viral nucleic acid in the cytoplasm after infection and transcription from the replicated multicopy nucleic acid.

Deleting the F gene in the Sendai virus vector can suppress the production of infectious virus particles from gene-introduced cells. Introducing a mutation into at least one of the L and P genes into the Sendai virus vector can make the vector temperature-sensitive.

Examples of the temperature-sensitive (TS) mutation in Sendai virus include TS7 (Y942H/L1361C/L1558I mutation in L protein), TS12 (D433A/R434A/K437A mutation in P protein), TS13 (D433A/R434A/K437A mutation in P protein and L1558I mutation in L protein), TS14 (D433A/R434A/K437A mutation in P protein and L1361C mutation in L protein), and TS15 (D433A/R434A/K437A mutation in P protein and L1361C/L1558I mutation in L protein).

The Sendai virus vector is, for example, an F gene-deleted (ΔF) Sendai virus vector that has G69E, T116A, and A183S mutations in M protein, A262T, G264R, and K461G mutations in HN protein, L511F mutation in mutations in P protein, N1197S and K1795E mutations in L protein and has also TS7, TS12, TS13, TS14, or TS15 mutation as described above. However, the temperature-sensitive mutations of the Sendai virus vector are not limited to these.

Examples of the Sendai virus vectors include SeV(PM)/TSΔF, SeV18+/TSΔF, or SeV(HNL)/TSΔF that has TS7, TS12, TS13, TS14, or TS15 mutation as described above. However, the temperature-sensitive mutations of Sendai virus vectors are not limited to these.

The Sendai virus vector to be introduced into cells may be a combination of a temperature-sensitive Sendai virus vector and a temperature-insensitive Sendai virus vector. Alternatively, the Sendai virus vector to be introduced into cells is a temperature-sensitive Sendai virus vector alone and is not required to include a temperature-insensitive Sendai virus vector. For example, the Sendai virus vector to be introduced into the cells is a temperature-sensitive Sendai virus vector alone into which TS7, TS12, TS13, TS14, or TS15 mutation has been introduced and is not required to include a temperature-insensitive Sendai virus vector. For example, the Sendai virus vector to be introduced into the cells is a Sendai virus vector alone that has temperature sensitivity equal to or greater than that of a temperature-sensitive Sendai virus vector into which TS7, TS12, TS13, TS14, or TS15 mutation has been introduced, and is not required to include a temperature-insensitive Sendai virus vector. For example, the Sendai virus vector to be introduced into the cells is a Sendai virus vector alone that has temperature sensitivity equal to or greater than that of a temperature-sensitive Sendai virus vector into which TS7, TS12, TS13, TS14, or TS15 mutation has been introduced, and is not required to include a Sendai virus vector that has temperature sensitivity less than that of a temperature-sensitive Sendai virus vector into which TS7, TS12, TS13, TS14, or TS15 mutation has been introduced.

The Sendai virus vector to be introduced into cells carry any reprogramming factors. The Sendai virus vector to be introduced into cells is, for example, a combination of a temperature-sensitive Sendai virus vector that comprises KLF RNA, OCT RNA, and SOX RNA in this order but does not comprise MYC RNA and another temperature-sensitive Sendai virus vector that comprises MYC RNA but does not comprise KLF RNA, OCT RNA, and SOX RNA. However, the number, combination, and order of reprogramming factors carried by the Sendai virus vector are arbitrary and are not particularly limited.

The Sendai virus vector to be introduced into cells may include a Sendai virus vector that comprises KLF RNA but does not comprise OCT RNA and SOX RNA. The Sendai virus vector that comprises KLF RNA but does not comprise OCT RNA and SOX RNA may be temperature-sensitive or temperature-insensitive.

The temperature-sensitive Sendai virus vector that comprises KLF RNA, OCT RNA, and SOX RNA is, for example, an F gene-deleted Sendai virus vector that has G69E, T116A, and A183S mutations in M protein, A262T, G264R, and K461G mutations in HN protein, L511F mutation in P protein, and N1197S and K1795E mutations in L protein and has also TS7, TS12, TS13, TS14, or TS15 mutation as described above. The temperature-sensitive mutation is, for example, TS7 or TS12, or TS12.

Examples of the temperature-sensitive Sendai virus vector that comprises KLF RNA, OCT RNA, and SOX RNA include SeV(PM)KOS/TS7ΔF, SeV(PM)KOS/TS12ΔF, and SeV(PM)KOS/TS12ΔF.

Examples of the temperature-sensitive Sendai virus vector that comprises MYC RNA include an F gene-deleted Sendai virus vector that has G69E, T116A, and A183S mutations in M protein, A262T, G264R, and K461G mutations in HN protein, L511F mutation in P protein, and N1197S and K1795E mutations in L protein and has also TS7, TS12, TS13, TS14, or TS15 mutation as described above. The temperature-sensitive mutation is TS15, for example.

Examples of the temperature-sensitive Sendai virus vector that comprises MYC RNA include SeV(HNL)MYC/TS12ΔF, SeV(HNL)MYC/TS13ΔF, SeV(HNL)MYC/TS15ΔF, and SeV(HNL)MYC/TS15ΔF.

Examples of the Sendai virus vector that comprises KLF RNA but does not comprise OCT RNA and SOX RNA include an F gene-deleted Sendai virus vector that has G69E, T116A, and A183S mutations in M protein, A262T, G264R, and K461G mutations in HN protein, L511F mutation in P protein, and N1197S and K1795E mutations in L protein. The Sendai virus vector that comprises KLF RNA but does not comprise OCT RNA and SOX RNA is, for example, less temperature-sensitive than the Sendai virus vector that comprises TS7, TS12, TS13, TS14, or TS15 mutation as described above and can express KLF gene even at a temperature equal to or higher than the predetermined temperature.

Examples of the Sendai virus vector that comprises KLF RNA but does not comprise OCT RNA and SOX RNA include SeV18+KLF4/TSΔF.

When a plurality of Sendai virus vectors are introduced into cells, for example, the Sendai virus vectors are introduced into cells simultaneously. Alternatively, it is preferable that a Sendai virus vector is introduced into the cells, followed by all of the remaining Sendai virus vectors within 48 hours.

The multiplicity of infection (MOI) of the Sendai virus vector used to infect cells is, for example, 0.1 or greater. For example, the MOI is equal to or less than 100.

The temperature used to infect cells with a Sendai virus vector may be below a predetermined temperature at which a viral nucleic acid of a temperature-sensitive Sendai virus vector is less stable. In other words, the temperature may be a temperature at which the viral nucleic acid of the temperature-sensitive Sendai virus vector is stable or may be equal to or higher than the predetermined temperature. When the Sendai virus vector is a temperature-sensitive Sendai virus vector alone and does not include a temperature-insensitive Sendai virus vector, the temperature used to infect cells with the Sendai virus vector is preferably below the predetermined temperature at which the viral nucleic acid of the temperature-sensitive Sendai virus vector is less stable. In other words, the temperature is preferably a temperature at which the viral nucleic acid of the temperature-sensitive Sendai virus vector is stable.

The cells to be introduced with reprogramming factors may be cultured on a substrate or in suspension.

Somatic cells to be introduced with reprogramming factors may be cultured in the absence of feeders using basement membrane matrix such as Matrigel (Corning), CELLstart® (ThermoFisher), Laminin511 (iMatrix-511, Nippi), fibronectin, or vitronectin.

A medium that can be used to culture the cells to be introduced with reprogramming factors includes, for example, a medium for stem cells including human ES/iPS cells, such as Primate ES Cell Medium (ReproCELL), Stemfit AK02N, Stemfit AK03 (Ajinomoto), or TeSR-E8 (STEMCELL Technologies). The medium for stem cells is placed in a culture vessel such as a dish, a well, or a tube.

After the cells are infected with the Sendai virus vector, the cells may be cultured for at least 2 days or from 2 to 10 days at a temperature lower than a predetermined temperature at which the viral nucleic acid of the temperature-sensitive Sendai virus vector is less stable, or at a temperature at which the viral nucleic acid of the temperature-sensitive Sendai virus vector is stable. The cells may be then cultured at or above the predetermined temperature. The medium may be replaced, for example, once every two days while the cells are cultured at or above the predetermined temperature.

After the cells are infected with the Sendai virus vector, the cells may be cultured for at least 2 days, or from 2 to 10 days, at a temperature that is equal to or greater than 4.0° C. and is lower than 37.0° C., for example. Subsequently, the temperature may be increased to culture the cells at a temperature that is equal to or higher than 36.5° C. and equal to or lower than 40.0° C. The temperature may be increased at once or in stages. After the temperature is increased, the medium may be replaced, for example, once every two days during cell culture.

After the cells are infected with the Sendai virus vector, the cells may be cultured at a temperature lower than a predetermined temperature at which the viral nucleic acid of the temperature-sensitive Sendai virus vector is less stable, or at a temperature at which the viral nucleic acid of the temperature-sensitive Sendai virus vector is stable, until stem cell-like colonies begin to appear. After the stem cell-like colonies begin to appear, the cells may be cultured at or above the predetermined temperature. The medium may be replaced, for example, once every two days while the cells are cultured at or above the predetermined temperature.

After the cells are infected with the Sendai virus vector, the temperature is increased, and the cells may be cultured at a temperature that is, for example, equal to or higher than 4° C. and lower than 37.0° C. until stem cell-like colonies begin to appear. After the stem cell-like colonies begin to appear, the cells may be cultured at a temperature that is equal to or higher than 36.5° C. and equal to or lower than 40.0° C. by increasing the temperature. The temperature may be increased at once or in stages. After the temperature is increased, the medium may be replaced, for example, once every two days during cell culture.

After the cells are introduced with reprogramming factors and then cultured, the cells that have been introduced with reprogramming factors are collected, and at least some of the collected and mixed-up cells are passaged at least once by seeding them into a medium. When the cells are passaged, clones of the cells that have been introduced with reprogramming factors may be mixed together. When the cells are passaged, different clones of the cells that have been introduced with reprogramming factors may be mixed together. The cells that have been introduced with reprogramming factors are then collected, and at least some of the collected and mixed-up cells are passaged multiple times by seeding them into a medium. The cells that have been introduced with reprogramming factors may be collected, and at least some of the collected and mixed-up cells may be passaged by seeding them into a medium until stem cells are established. It should be noted that all of the collected and mixed-up cells may be seeded into a medium.

The phrase “the cells that have been introduced with reprogramming factors are collected, and at least some of the collected and mixed-up cells are passaged by seeding them into a medium” as used herein refers to, for example, passaging the cells that have been introduced with reprogramming factors without distinguishing the cells based on their gene expression states. For example, when passaged, the cells that have been introduced with reprogramming factors may be seeded in the same culture vessel without distinguishing the cells based on their gene expression states. Alternatively, the phrase “the cells that have been introduced with reprogramming factors are collected, and at least some of the collected and mixed-up cells are passaged by seeding them into a medium” as used herein refers to, for example, passaging the cells that have been introduced with reprogramming factors without distinguishing the cells based on the degree of reprogramming. For example, when passaged, the cells that have been introduced with reprogramming factors may be seeded in the same culture vessel without distinguishing the cells based on the degree of reprogramming.

Alternatively, the phrase “the cells that have been introduced with reprogramming factors are collected, and at least some of the collected and mixed-up cells are passaged by seeding them into a medium” as used herein refers to, for example, passaging the cells that have been introduced with reprogramming factors without distinguishing the cells based on their morphology. For example, when passaged, the cells that have been introduced with reprogramming factors may be seeded in the same culture vessel without distinguishing the cells based on their morphology. Alternatively, the phrase “the cells that have been introduced with reprogramming factors are collected, and at least some of the collected and mixed-up cells may be passaged by seeding them into a medium” as used herein refers to, for example, passaging the cells that have been introduced with reprogramming factors without distinguishing the cells based on their size. For example, when passaged, the cells that have been introduced with reprogramming factors may be seeded in the same culture vessel without distinguishing the cells based on their size.

Alternatively, the phrase “the cells that have been introduced with reprogramming factors are collected, and at least some of the collected and mixed-up cells are passaged by seeding them into a medium” as used herein refers to passaging the cells that have been introduced with reprogramming factors without cloning the cells. For example, when the cells are passaged without cloning, colonies formed by the cells that have been introduced with reprogramming factors are not required to be picked up. For example, when the cells are passaged without cloning, multiple colonies formed by the cells that have been introduced with reprogramming factors are not required to be separated from each other. For example, when passaged, the cells that have formed multiple different colonies may be mixed together and then seeded in the same culture vessel. For example, when the cells are passaged without cloning, a single colony formed by the cells that have been introduced with reprogramming factors is not required to be cloned. For example, when the cells are passaged, the colonies may be mixed together and then seeded in the same culture vessel.

For example, when cells that have been introduced with reprogramming factors are adhesion-cultured on a substrate, the cells cultured on the substrate are collected, and at least some of the collected and mixed-up cells may be passaged by seeding them into a medium. For example, when passaged, the cells are detached from the culture vessel, and at least some of the detached and mixed-up cells may be passaged by seeding them in the same vessel. For example, the cells are detached from the culture vessel with a detachment solution, and all of the detached and mixed-up cells may be passaged. For example, cells that have not formed colonies may be passaged. When the cells that have been introduced with reprogramming factors are cultured in suspension, all of the cells cultured in suspension may be passaged.

When the cells that have been introduced with reprogramming factors are passaged, the cells may be seeded in a medium or culture vessel at a low density. The low density as used herein refers to, for example, 1 cell/cm² or more and 0.25×10⁴ cells/cm² or less, 1.25×10³ cells/cm² or less, 0.25×10³ cells/cm² or less, 0.25×10² cells/cm² or less, or 0.25×10¹ cells/cm² or less. Alternatively, the low density refers to a density at which 10 cells or fewer, 9 cells or fewer, 8 cells or fewer, 7 cells or fewer, 6 cells or fewer, 5 cells or fewer, 4 cells or fewer, 3 cells or fewer, or 2 cells or fewer can contact each other, and 11 cells or more does not contact each other. It should be noted that multiple cell clusters in which 10 cells or more contact each other may be found. Alternatively, when a state in which the bottom of cell vessel is entirely covered with cells is considered as 100% confluence, the low density refers to 5% confluence or less, 4% confluence or less, 3% confluence or less, 2% confluence or less, 1% confluence or less, 0.5% confluence or less, 0.1% confluence or less, 0.05% confluence or less, or 0.01% confluence or less. Alternatively, the low density refers to, for example, a density at which each of the seeded cells does not contact each other. For example, a single cell may be seeded in a well of a well plate. The well plate may be a 12-well plate or a 96-well plate. According to the findings of the present inventors, when cells that have been introduced with reprogramming factors are passaged, seeding the cells in a medium at a low density allows the suppression of persistence of Sendai virus in pluripotent stem cells that will be induced from the cells. The percentage of cells with persistent Sendai virus in induced pluripotent stem cells is, for example, 4% or less, 3% or less, 2% or less, 1% or less, 0.5% or less, or 0%.

When a temperature-sensitive Sendai virus vector is used, cells may be passaged and then cultured at a temperature equal to or higher than a predetermined temperature at which the viral nucleic acid of the temperature-sensitive Sendai virus vector is less stable. The cells are passaged and then cultured, for example, at a temperature that is equal to or higher than 36.5° C. and lower than 38.0° C. The cells are passaged and then cultured, for example, at a temperature that is equal to or higher than 36.5° C. and lower than 38.0° C. until cell-cell adhesion begins. Once cell-cell adhesion begins, the cells may be cultured at a temperature that is higher than that used until cell-cell adhesion begins, for example, at a temperature that is equal to or higher than 37.5° C. and lower than 42.0° C. The cells may be also passaged and then cultured at a temperature that is equal to or higher than 37.5° C. and equal to or lower than 42.0° C. from before the beginning of cell-cell adhesion.

The cells that have been introduced with reprogramming factors may be cultured in a closed culture vessel and passaged. The closed culture vessel allows no exchange of, for example, gas, viruses, microorganisms, impurities, and the like between the inside space of the vessel and external environment. The cells that have been introduced with reprogramming factors may be expanded in two- or three-dimensional culture.

After the cells that have been introduced with reprogramming factors are induced into pluripotent stem cells, and the pluripotent stem cells are established, all of the cells cultured on a substrate may be cryopreserved as pluripotent stem cells. For example, all of the cells are detached from the culture vessel with a detachment solution may be cryopreserved as pluripotent stem cells. After the cells that have been introduced with reprogramming factors are induced into pluripotent stem cells, all of the cells cultured in suspension may be cryopreserved as pluripotent stem cells.

The induced pluripotent stem cells can form flat colonies similar to those of ES cells and can express alkaline phosphatase. The induced pluripotent stem cells can express undifferentiated cell markers, such as Nanog, OCT4, and SOX2. The induced pluripotent stem cells can express TERT. The induced pluripotent stem cells can exhibit telomerase activity.

Whether the cells have been induced into pluripotent stem cells or not may also be determined by analyzing whether the cells are positive for at least one surface marker selected from TRA-1-60, TRA-1-81, SSEA-1, and SSEA5, which are cell surface markers showing that the cell is undifferentiated, on a flow cytometer. TRA-1-60 is an antigen specific to iPS/ES cells. iPS cells are induced only from a TRA-1-60-positive fraction, and thus TRA-1-60-positive cells can be considered as iPS cell species.

The method of introducing, into cells, inducers for induction into oligodendrocytes is not particularly limited. The inducers are introduced into cells, for example, by electroporation. Alternatively, the inducers are introduced into cells by, for example, lipofection. Alternatively, the inducers are introduced into cells using, for example, a viral vector. The inducers may be introduced into cells in an integration-free manner.

Cells to be introduced with inducers for induction into oligodendrocytes may be cultured on a substrate or in suspension.

Somatic cells to be introduced with inducers for induction into oligodendrocytes may be cultured in the absence of feeders using basement membrane matrix such as Matrigel (Corning), CELLstart® (ThermoFisher), Laminin511 (iMatrix-511, Nippi), fibronectin, and vitronectin.

A medium that can be used to culture the cells to be introduced with inducers for induction into oligodendrocytes includes, for example, media for stem cells such as Stem fit (AJINOMOTO CO., INC.) and mTeSR (Stem cell technologies).

When the cells introduced with the inducers are cultured in suspension or in three-dimensional culture, for example, a gel medium is used. The gel medium is prepared, for example, by adding gellan gum to a medium for stem cells.

The gel medium may contain at least one macromolecule compound selected from the group consisting of gellan gum, hyaluronic acid, rhamsan gum, diutan gum, xanthan gum, carrageenan, fucoidan, pectin, pectic acid, pectinic acid, heparan sulfate, heparin, heparitin sulfate, keratosulfate, chondroitin sulfate, dermatan sulfate, rhamnan sulfate, and salts thereof. The gel medium may also contain methylcellulose. Containing methylcellulose will further suppress cell aggregation.

Alternatively, the gel medium may contain at least one of temperature-sensitive gel selected from poly(glycerol monomethacrylate) (PGMA), poly(2-hydroxypropyl methacrylate) (PHPMA), poly(N-isopropylacrylamide) (PNIPAM), amine terminated, carboxylic acid terminated, maleimide terminated, N-hydroxysuccinimide (NHS) ester terminated, and triethoxysilane terminated, poly(N-isopropylacrylamide-co-acrylamide), poly(N-isopropylacrylamide-co-acrylic acid), poly(N-isopropylacrylamide-co-butylacrylate), poly(N-isopropylacrylamide-co-methacrylic acid), poly(N-isopropylacrylamide-co-methacrylic acid-co-octadecyl acrylate), and N-isopropylacrylamide.

The gel medium may or may not contain a growth factor such as basic fibroblast growth factor (bFGF). Alternatively, the gel medium may contain a growth factor such as bFGF at a low concentration of 400 μg/L or less, 40 μg/L or less, or 10 μg/L or less.

The gel medium may contain TGF-β, does not contain TGF-β, or may contain TGF-β at a low concentration of 600 μg/L or less, 300 μg/L or less, or 100 μg/L or less.

The gel medium is not required to be stirred. The gel medium also is not required to contain feeder cells.

The gel medium may contain at least one substance selected from the group consisting of cadherin, laminin, fibronectin, and vitronectin.

The method for producing oligodendrocytes and progenitor cells thereof according to the embodiment can induce cells into oligodendrocytes, for example, within 30 days, 20 days, 10 days, or 7 days after the cells are introduced with inducers. Immature oligodendrocytes like oligodendrocyte progenitor cells and mature oligodendrocytes express at least one selected from O4 and PLP (Myelin Proteolipid Protein) 1. Mature oligodendrocytes express myelin oligodendrocyte glycoprotein (MOG) and platelet-derived growth factor receptor (PDGFR) α.

### EXAMPLES

### Examples 1 and 2

Laminin 511-coated dishes were used as dishes for inducing pluripotent stem cells. Human peripheral blood mononuclear cells were suspended in a blood medium and counted with a hemacytometer to adjust the number of mononuclear cells in the blood medium. The mononuclear cells were then cultured on dishes for inducing pluripotent stem cells in two-dimensional culture at 37° C. for 1 to 7 days.

To the mononuclear cells being cultured in two-dimensional culture were added SeV(PM)hKOS/TS12ΔF and SeV(HNL)hC-Myc/TS15ΔF at an MOI of 5. The dishes for inducing pluripotent stem cells were then placed in an incubator at 34° C. to culture the cells. Two days after the infection, the blood medium was replaced with a medium for iPS cells. The medium was then replaced with the medium for iPS cells once every 2 days. During culturing, the temperature was elevated stepwise to 37° C. and 38° C.

Eight days after infection, stem cell-like cell clusters appeared. Almost all of the cells were positive for TRA1-60 on day 14 after infection and exhibited iPS cell-like morphology. Fourteen days after infection, a cell detachment agent TrypLE Select was added to the dishes and allowed to stand at room temperature for 1 minute. The suspension comprising the cells was then aspirated and incubated at 37° C. for 5 to 10 minutes. A medium for iPS cells was then added, and the medium comprising the cells was collected in a 15 mL tube. The cells were counted with a hemacytometer to adjust the concentration of the suspension comprising the cells. The cells were seeded in a well plate at a concentration equal to or less than 0.25×10⁴ cells/cm² for the first passage. In the first passage of Example 1, the cells were all detached from the well plate, and the detached and mixed-up cells were seeded in the next well plate without distinction. In contrast, in the first passage of Example 2, colonies were picked up and cloned. It should be noted that in both Examples 1 and 2, the cells were seeded not to contact 11 cells or more with each other when passaged.

Next, the well dish was placed in an incubator at 37° C. to culture the cells in two-dimensional culture. After the cells began to divide, the culture temperature was elevated to 38° C. In both Examples 1 and 2, all of the cells were then collected every time the cells were 60% to 80% confluent, and at least some of the collected and mixed-up cells were passaged by seeding them into the medium. In the second and subsequent passages, the cells were also seeded in a well plate at a density of 0.25×10⁴ cells/cm² or less. Again, 11 cells or more did not contact with each other.

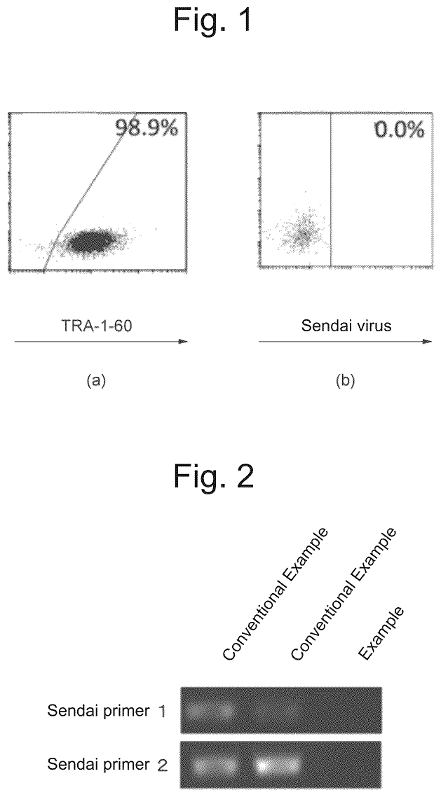

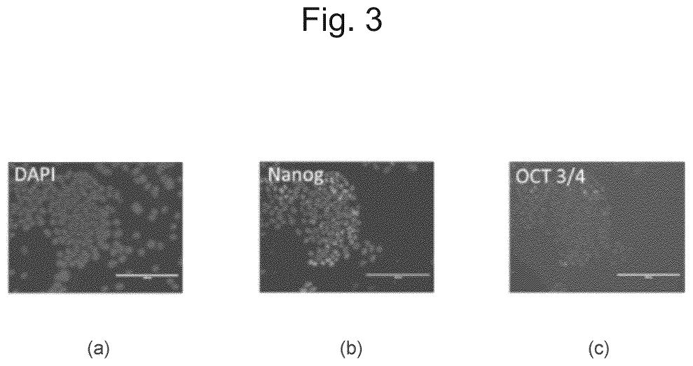

As shown in FIG. 1, cells that had been passaged only once were stained with an anti-Sendai virus antibody. Sendai virus remaining in the cells according to Example 1 was evaluated with a flow cytometer, and Sendai virus in the cells disappeared. As shown in FIG. 2, no Sendai virus remaining in the cells according to Example 1 was also detected by PCR. When the cells were seeded at a high density causing 11 cells or more to adhere to each other, as in the conventional procedure, Sendai virus remained in the cells. Immunostaining images of the resulting TRA1-60-positive cells are shown in FIG. 3.

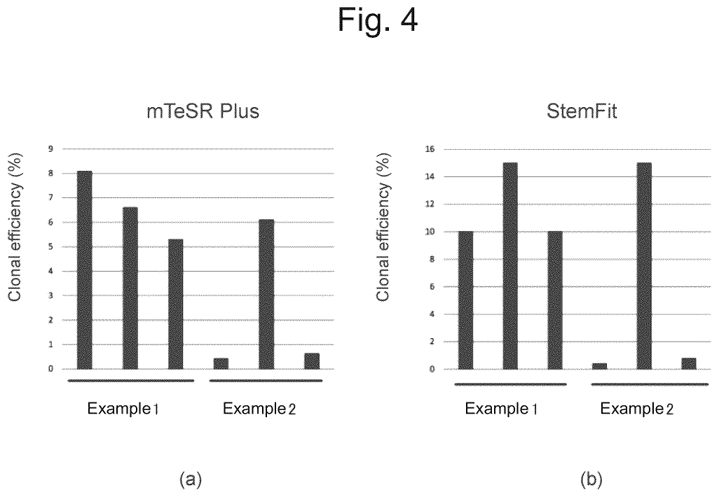

The number of colonies formed was also counted five days after the disappearance of Sendai virus in the cells. In addition, the number of colonies was divided by the number of cells seeded to calculate clonal efficiency. The triplicate results are shown in FIG. 4. When all of the cells were collected in the first passage using mTeSR Plus as a medium, and some of the collected and mixed-up cells were passaged by seeding them in the medium, the clonal efficiency ranged from about 5% to about 8%, with a small variation. When the colonies were picked up and cloned in the first passage using mTeSR Plus as a medium, the clonal efficiency varied, with about 6% or less than 1%. When all of the cells were collected in the first passage using StemFit as a medium, and some of the collected and mixed-up cells were passaged by seeding them in the medium, the clonal efficiency ranged from about 10% to about 15%, with a small variation. When the colonies were picked up and cloned in the first passage using StemFit as a medium, the clonal efficiency varied, with about 16% or less than 1%.

Thus, it was shown that clonal efficiency is higher and more stable when all of the cells introduced with reprogramming factors were collected in the first passage, and at least some of the collected and mixed-up cells were passaged by seeding them in the medium.

### Example 3

A well dish coated with a solubilized basement membrane preparation (Matrigel, Corning) was prepared, and a feeder-free medium (mTeSR®1, Stemcell Technologies) supplemented with 10 nmol/mL of Rho-associated coiled-coil forming kinase/Rho-binding kinase (ROCK) inhibitor (Selleck) was added to each well.

Human iPS cells were dispersed in a detachment/dissociation/dispersion solution for tissues and cultured cells (Accutase, Innovative Cell Technologies) and seeded in the well dish. The iPS cells were then cultured in the feeder-free medium for 24 hours under a gas condition of 5% carbon dioxide and 20% oxygen.

The iPS cells were detached with a detachment agent from wells to prepare a single cell suspension. Next, the iPS cells were introduced with OLIG2, SOX10, ASCL1, and NKX2.2 as inducers by electroporation using a Human Stem Cell Nucleofector Kit® (LONZA) and an episomal plasmid. The cells introduced with the inducers were then seeded in a well dish and cultured in mTeSR.

On day 3 after introducing the inducers into the cells, a medium for oligodendrocytes was added to the wells. The medium for oligodendrocytes was prepared from DMEM/F12, N2(1×), B27(1×), penicillin-streptomycin (1×), NEAA (1×), insulin (25 μg/mL, Sigma), PDGF-AA (10 ng/ml, PeproTech), IGF (10 ng/mL, PeproTech), NT3 (1 ng/mL, PeproTech), biotin (100 ng/mL, Sigma), and cAMP (1 μmol/L, Sigma). On day 5, the medium for oligodendrocytes was also added to the wells. On day 7, the medium in the wells was all replaced with a fresh medium for oligodendrocytes. Human fibroblast cells or human glial cells introduced with the inducers were then seeded and cultured until day 21. The cells were stained with an anti-O4 antibody on day 7 after being introduced with the inducers. The cells were stained with an anti-O4 antibody, an anti-PLP1 antibody, and an anti-MOG antibody on day 21 after introduced with the inducers.

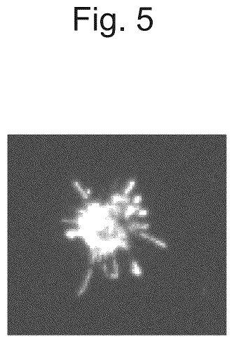

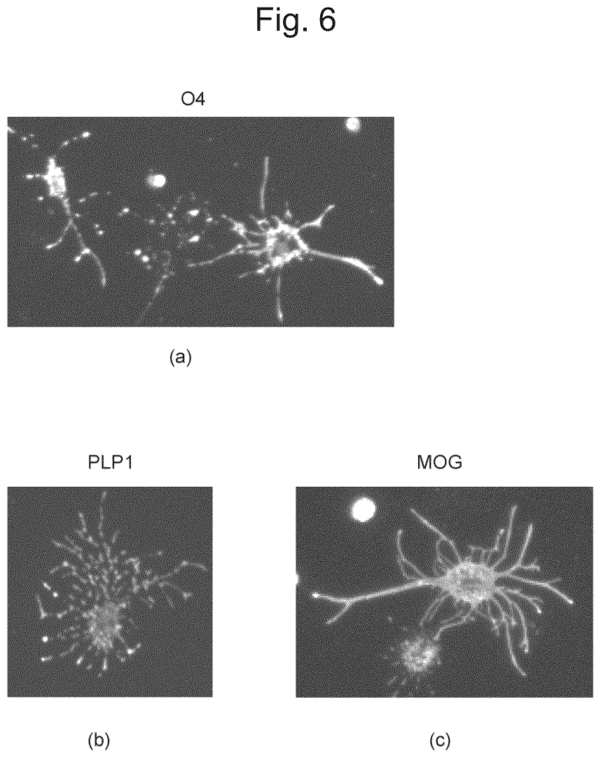

As shown in FIG. 5, the cells on day 7 after being introduced with the inducers expressed O4, which is a marker for oligodendrocytes and progenitor cells thereof. As shown in FIG. 6, the cells on day 21 after being introduced with the inducers expressed O4, PLP1, and MOG. Whether the iPS cells introduced with the inducers were prepared in Example 1 or 2, the expression of the markers for oligodendrocytes and progenitor cells thereof was confirmed.

### Example 4

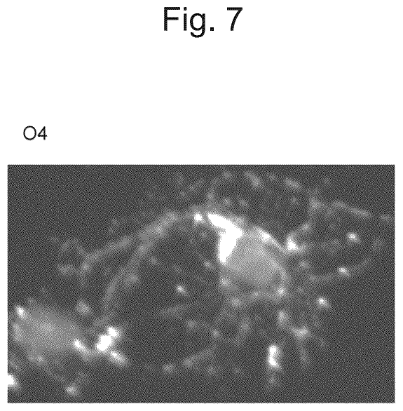

iPS cells were introduced with inducers as in Example 3, except the inducers were OLIG2, SOX10, ASCL1, and NKX6.1. As shown in FIG. 7, the cells introduced with the inducers expressed O4.

### Example 5

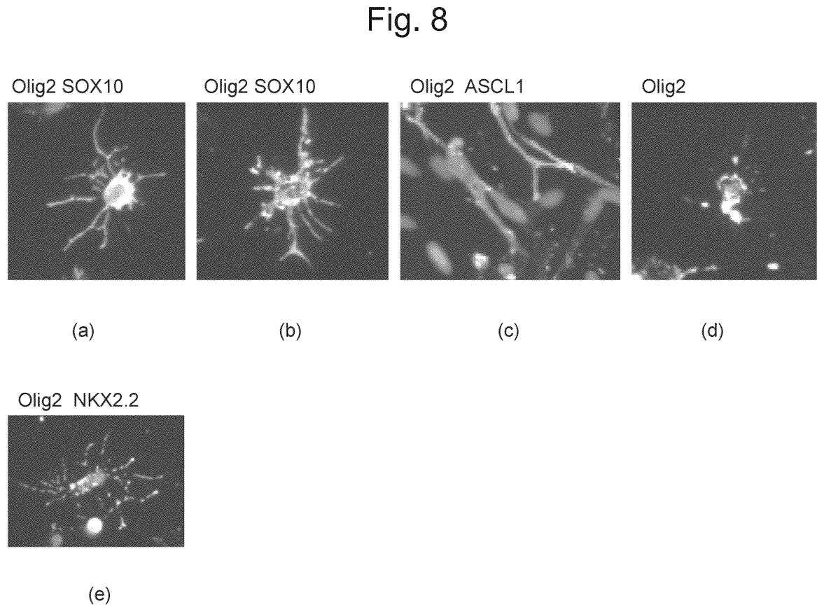

iPS cells were introduced with inducers as in Example 3, except the inducers were OLIG2, SOX10, and ASCL1. As shown in FIG. 8(a), the cell introduced with the inducers expressed O4.

### Example 6

iPS cells were introduced with inducers as in Example 3, except the inducers were OLIG2 and SOX10. As shown in FIG. 8(b), the cell introduced with the inducers expressed O4.

### Example 7

iPS cells were introduced with inducers as in Example 3, except the inducers were OLIG2 and ASCL1. As shown in FIG. 8(c), the cells introduced with the inducers expressed O4.

### Example 8

iPS cells were introduced with an inducer as in Example 3, except the inducer was OLIG2. As shown in FIG. 8(d), the cell introduced with the inducers expressed O4.

### Example 9

iPS cells were introduced with inducers as in Example 3, except the inducers were OLIG2 and NKX2.2. As shown in FIG. 8(e), the cell introduced with the inducers expressed O4, which is a marker for oligodendrocytes and progenitor cells thereof.

### Example 10

A well dish coated with a solubilized basement membrane preparation (Matrigel, Corning) was prepared, and a medium was added to each well. Furthermore, adult human peripheral blood mononuclear cells separated with Ficoll (GE) were seeded in the well dish. The mononuclear cells were then cultured in a blood medium (StemSpan®, SFEM II, STEMCELL TECHNOLOGIES) at 37° C. for 1 to 7 days. The medium contained 20 ng/mL FLT3, 10 ng/mL TPO, 50 ng/mL IL6, 10 ng/mL GCSF, 50 ng/mL SCF, 20 ng/mL IL3, and 10 ng/mL GM-CSF. The cells were cultured on the dish under a gas condition of 5% carbon dioxide and 20% oxygen.

The mononuclear cells were collected from the wells to prepare a single cell suspension. Next, the mononuclear cells were introduced with OLIG2, SOX10, ASCL1, and NKX2.2 as inducers by electroporation using a Nucleofector kit for CD34+ Cells® (LONZA) and an episomal plasmid. The cells introduced with the inducers were then seeded in a well dish and cultured in the blood medium.

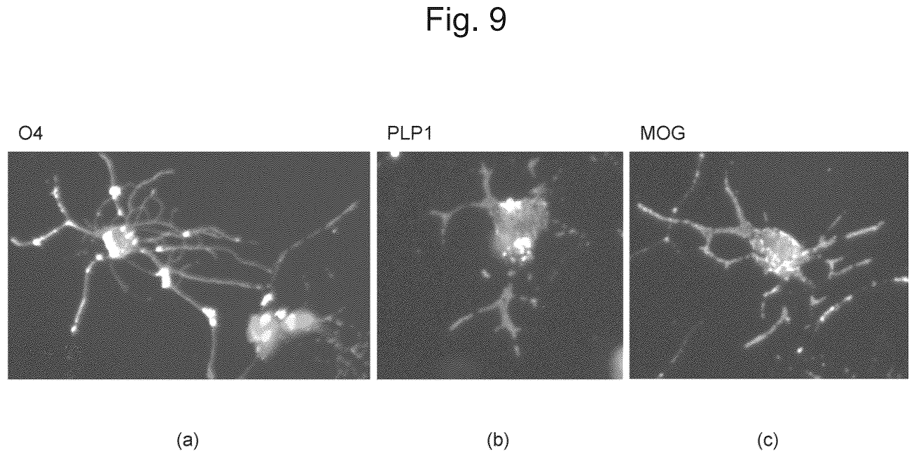

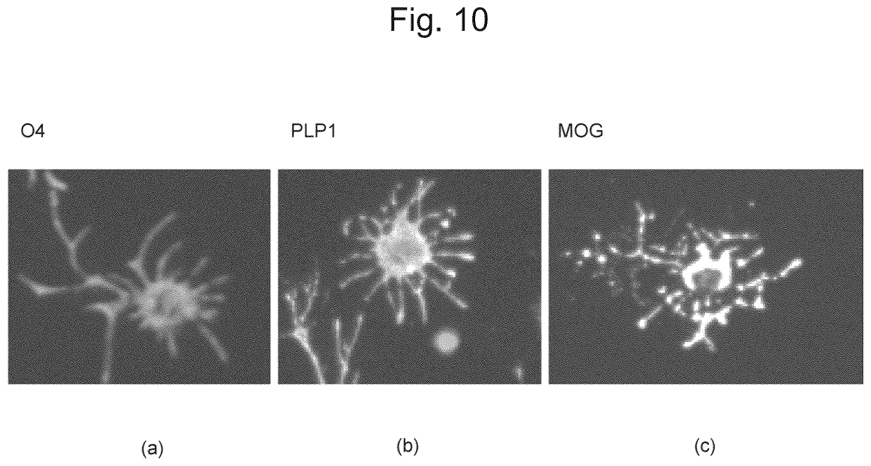

On day 3 after introducing the inducers into the cells, a medium for oligodendrocytes was added to the wells. On day 5, the medium for oligodendrocytes was also added to the wells. On day 7, the medium in the wells was all replaced with a fresh medium for oligodendrocytes. Human fibroblast cells introduced with the inducers were then seeded and cultured until day 28. The cells were stained with an anti-O4 antibody, an anti-PLP1 antibody, or an anti-MOG antibody on day 28 after being introduced with the inducers. As shown in FIG. 9, the cells on day 28 after being introduced with the inducers expressed O4, PLP1, and MOG.

### Example 11

A well dish coated with a solubilized basement membrane preparation (Matrigel, Corning) was prepared, and a medium (DMEM containing 10% FBS) was added to each well. Human neonatal fibroblast cells were further seeded in the well dish. The fibroblast cells were then cultured on the dish at 37° C. for 1 to 7 days under a gas condition of 5% carbon dioxide and 20% oxygen.

The fibroblast cells were detached with a detachment agent from wells to prepare a single cell suspension. Next, the fibroblast cells were introduced with OLIG2, SOX10, ASCL1, and NKX2.2 as inducers by electroporation using a Human Dermal Fibroblast Nucleofector Kit® (LONZA) and an episomal plasmid. The cells introduced with the inducers were then seeded in a well dish and cultured in DMEM medium containing 10% FBS.

On day 3 after introducing the inducers into the cells, a medium for oligodendrocytes was added to the wells. On day 5, the medium for oligodendrocytes was also added to the wells. On day 7, the medium in the wells was all replaced with a fresh medium for oligodendrocytes. Human fibroblast cells introduced with the inducers were then seeded and cultured until day 28. The cells were stained with an anti-O4 antibody, an anti-PLP1 antibody, or an anti-MOG antibody on day 28 after being introduced with the inducers. As shown in FIG. 10, the cells on day 28 after being introduced with the inducers expressed O4, PLP1, and MOG.

### Example 12

A well dish coated with a solubilized basement membrane preparation (Matrigel, Corning) was prepared, and a medium was added to each well. Adult human peripheral blood T cells were further seeded in the well dish. The T cells were then expanded on the dish at 37° C. for 1 to 7 days under a gas condition of 5% carbon dioxide and 20% oxygen. It should be noted that the expand culture is not necessarily required. A serum-free medium (X-VIV010, Lonza) supplemented with 30 U/mL interleukin 2 and beads for proliferation and stimulation of human T cells (Dynabeas CD3/CD28, BD) was used.

The T cells were collected from the wells to prepare a single cell suspension. Next, the T cells were introduced with OLIG2, SOX10, ASCL1, and NKX2.2 as inducers by electroporation using a T cell electroporation Kit® (LONZA) and an episomal plasmid. The cells introduced with the inducers were then seeded in a well dish and cultured in a serum-free medium (X-VIVO10, Lonza) supplemented with 30 U/mL interleukin 2 and beads for proliferation and stimulation of human T cells (Dynabeas CD3/CD28, BD).

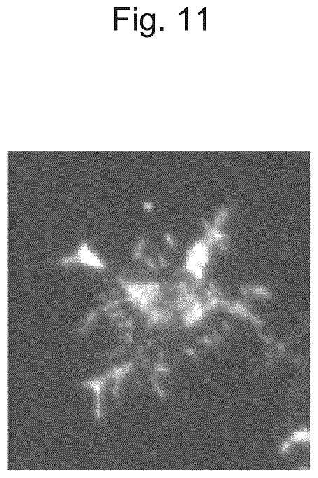

On day 3 after introducing the inducers into the cells, a medium for oligodendrocytes was added to the wells. On day 5, the medium for oligodendrocytes was added to the wells again. On day 7, the medium in the wells was all replaced with a fresh medium for oligodendrocytes. Human fibroblast cells introduced with the inducers were then seeded and cultured until day 28. The cells were stained with an anti-O4 antibody, an anti-PLP1 antibody, or an anti-MOG antibody on day 28 after being introduced with the inducers. As shown in FIG. 11, the cell on day 28 after being introduced with the inducers expressed O4, PLP1, and MOG.

### Example 13

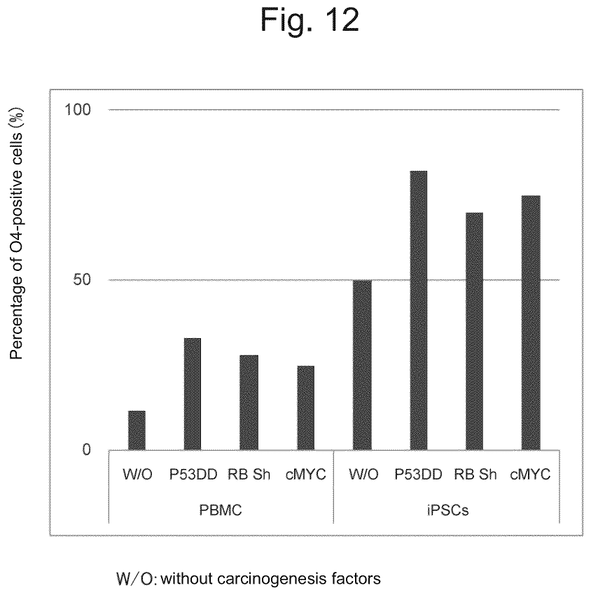

Oligodendrocytes were induced from human iPS cells as in Example 3, except p53DD was additionally used for the inducers. The results are shown in FIG. 12. The addition of p53DD increased the percentage of O4-positive cells.

### Example 14

Oligodendrocytes were induced from human iPS cells as in Example 3, except an shRNA for RB gene was additionally used for the inducers. The results are shown in FIG. 12. The addition of Rb Sh increased the percentage of O4-positive cells.

### Example 15

Oligodendrocytes were induced from human iPS cells as in Example 3, except c-MYC was additionally used for the inducers. The results are shown in FIG. 12. The addition of c-MYC increased the percentage of O4-positive cells.

### Example 16

Oligodendrocytes were induced from mononuclear cells derived from adult human peripheral blood as in Example 10, except p53DD was additionally used for the inducers. The results are shown in FIG. 12. The addition of p53DD increased the percentage of O4-positive cells.

### Example 17

Oligodendrocytes were induced from mononuclear cells derived from adult human peripheral blood as in Example 10, except RB Sh was additionally used for the inducers. The results are shown in FIG. 12. The addition of Rb Sh increased the percentage of O4-positive cells.

### Example 18

Oligodendrocytes were induced from mononuclear cells derived from adult human peripheral blood as in Example 10, except c-MYC was additionally used for the inducers. The results are shown in FIG. 12. The addition of c-MYC increased the percentage of O4-positive cells.

1. A method for producing oligodendrocytes and progenitor cells thereof, comprising introducing inducers including a combination of OLIG and SOX or a combination of OLIG and NKX into iPS or ES cells.

2. The method for producing oligodendrocytes and progenitor cells thereof according to claim 1, wherein the inducers comprise OLIG, NKX, and SOX.

3. The method for producing oligodendrocytes and progenitor cells thereof according to claim 1, wherein the inducers further comprise ASCL.

4. The method for producing oligodendrocytes and progenitor cells thereof according to claim 1, wherein the inducers further comprise a factor that promotes cell proliferation.

5.-21. (canceled)

22. A method for producing oligodendrocytes and progenitor cells thereof, comprising introducing inducers including OLIG, SOX, ASCL, and NKX into somatic cells.

23. The method for producing oligodendrocytes and progenitor cells thereof according to claim 22, wherein the inducers further comprise a factor that promotes cell proliferation.

24. A method for producing oligodendrocytes and progenitor cells thereof, comprising introducing inducers including OLIG and a factor that promotes cell proliferation into somatic cells.

25. The method for producing oligodendrocytes and progenitor cells thereof according to claim 24, wherein the inducers comprise at least any selected from the group consisting of SOX, ASCL, and NKX.

26. The method for producing oligodendrocytes and progenitor cells thereof according to claim 22, wherein the somatic cells are not iPS or ES cells.

27. The method for producing oligodendrocytes and progenitor cells thereof according to claim 22, wherein the somatic cells are blood cells.

28. The method for producing oligodendrocytes and progenitor cells thereof according to claim 22, wherein the somatic cells are mononuclear cells.

29. The method for producing oligodendrocytes and progenitor cells thereof according to claim 22, wherein the somatic cells are fibroblast cells.

30. The method for producing oligodendrocytes and progenitor cells thereof according to claim 24, wherein the somatic cells are not iPS or ES cells.

31. The method for producing oligodendrocytes and progenitor cells thereof according to claim 24, wherein the somatic cells are blood cells.

32. The method for producing oligodendrocytes and progenitor cells thereof according to claim 24, wherein the somatic cells are mononuclear cells.

33. The method for producing oligodendrocytes and progenitor cells thereof according to claim 24, wherein the somatic cells are fibroblast cells.
